(19) United States
(12) Patent Application Publication
(10) Pub. No.: US 2004/0092999 A1
(43) Pub. Date: May 13, 2004

(54) Post-withdrawal venipuncture bruising control device and method

(76) Inventor: Blazej Lojewski, Park Ridge, IL (US)

Correspondence Address: IP FOCUS LAW GROUP, LTD, 608 NORTH CARLYLE LANE, SUITE 100, ARLINGTON HEIGHTS, IL, 60004, US

(21) Appl. No.: 10/256,546

(22) Filed: Sep. 28, 2002

(51) Int. Cl. A61M 29/00

(52) U.S. Cl. 606/185

(57) ABSTRACT

An improved, user-friendly medical device is provided for inducing blood vessel distension and for reducing post-venipuncture bruising. The device comprises an elastomeric band adapted to be stretched to encircle a limb with overlap lengths, a pressure applicator secured to the band and a pair of complementary releasably engageable coupling pads spaced apart and secured to the elastomeric band. At least one of the pair of coupling pads is elastic such that the device is elastic over at least about 80 percent of the length of the band.

### FIELD OF THE INVENTION

This invention relates to a device to promote distention for vein spotting and to reduce post-withdrawal bruising in phlebotomy and other venipuncture procedures.

### BACKGROUND OF THE INVENTION

Medical procedures involving needle venipuncture typically begin with the use of a tourniquet device near the desired puncture site to distend one or more veins for easy visual identification by the attendant. The same procedures typically end when the attendant removes the puncture needle and applies finger, i.e. digital, pressure on the puncture site over a bandage.

For younger and more robust patients, a typical phlebotomy wound is stable for finger release after just a couple moments and the resulting puncture site is almost undetectable. For older more fragile patients, however, an extensive period of finger pressure is required to prevent bruising about the puncture site. While minor bruising is not a health concern, patients are known to complain about the appearance. Medical personnel are often forced to use otherwise productive time waiting for sufficient clotting at the venipuncture site.

Devices for unattended pressure application at a venipuncture site have been proposed. For example, U.S. Pat. No. 4,182,338 to Stanulis is directed to a device for providing localized pressure to a puncture wound. The device includes an elastomeric pyramid-shaped body attached to securing straps. U.S. Pat. No. 5,269,803 to Geary et al. is directed to housing attached to an adjustable band for tightening the housing against a puncture site before needle puncture. The Geary patent further describes leaving the pad in place after the needle is withdrawn to stop bleeding.

Such prior designs have thus far not been commercially successful. Venipuncture, especially for blood drawings, is a very routine procedure for medical attendants. Experienced phlebotomists favor neither new additional steps nor new additional equipment unless such extras are user friendly and immediately beneficial. The prior approaches to the hemostasis problem are neither user friendly nor compatible with existing venipuncture procedures.

For example, both of the prior art approaches to the hemostasis problem discussed above call for the medical attendant to have available a tourniquet and an extra device, the pressure applicator. Furthermore, the prior art devices include new materials or mechanisms not immediately familiar to medical personnel.

Accordingly, it would be desirable to provide a device that reduces post-venipuncture bruising without continuous user attention. Specifically, it would be desirable to provide a hemostasis aid that is user friendly and compatible with existing venipuncture procedures.

### SUMMARY OF THE INVENTION

Devices according to the present invention are suitable for both inducing blood vessel distension and for reducing post-venipuncture bruising. The devices comprise an open elastomeric band adapted to be stretched to encircle a limb with overlapping lengths, a pressure applicator secured to the band and a pair of complementary releasably engageable coupling pads spaced apart and secured to the elastomeric band. At least one of the pair of coupling pads is elastic such that the device is elastic over at least about 80 percent of the length of the band. The pair of complementary releasably engageable coupling pads preferably employ a multiminihook-to-pile adhesion system.

In a preferred embodiment at least 80 percent of the length of the band has a length-wise elasticity substantially the same as a conventional, disposable rubber tourniquet. And surprisingly, it has been found that, in addition to compatible tactile response, the location of the pressure applicator at one end of the elastomeric band is an important feature for gaining acceptance by phlebotomists.

### BRIEF DESCRIPTION OF THE DRAWINGS

In the accompanying drawings that form part of the specification, and in which like numerals are employed to designate like parts throughout the same,

FIG. 1 is a perspective view of a bruising control device according to the present invention;

FIG. 2 is a fragmentary cross-section of the pressure applicator taken along the plane 2-2 in FIG. 1;

FIG. 3 is a perspective view of a bruising control device according to an alternate embodiment of the present invention;

FIG. 4 is a fragmentary cross-section of the pressure applicator taken along the plane 4-4 in FIG. 3;

FIG. 5 is perspective view showing the use of the device of FIG. 1 for distending blood vessels;

FIG. 6 is a perspective view showing the use of the device of FIG. 1 for bruising control after a venipuncture procedure; and

FIG. 7 is a perspective view of a bruising control device according to an alternate embodiment of the present invention.

### DESCRIPTION OF THE PREFERRED EMBODIMENTS

While this invention is susceptible to embodiment in many different forms, this specification and the accompanying drawings disclose only preferred forms as examples of the invention. The invention is not intended to be limited to the embodiments so described, however. The scope of the invention is identified in the appended claims.

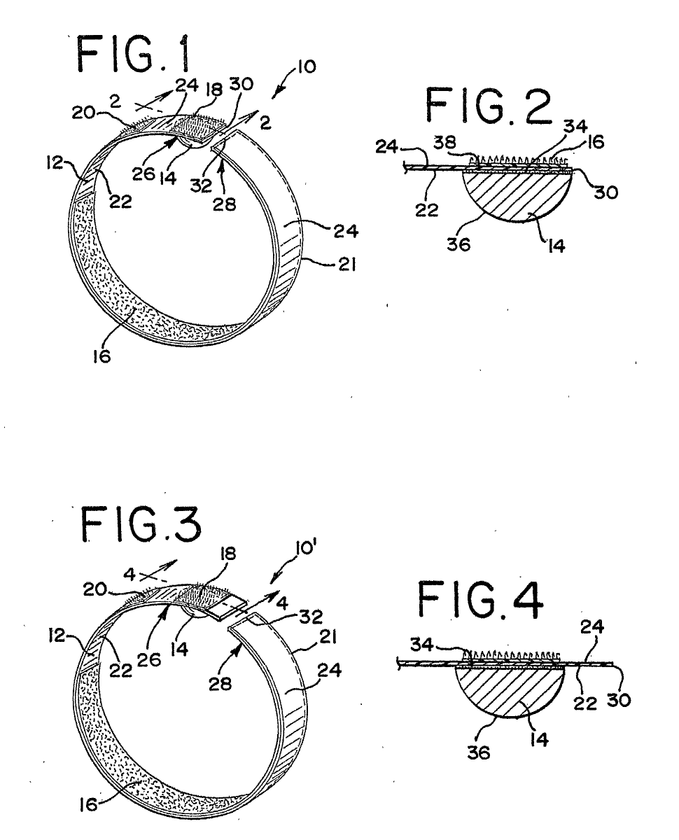

Referring to FIGS. 1 and 2, a hemostasis aid 10 includes an open band (or strip) of elastomeric material 12, a pressure applicator 14 and complementary coupling pads 16, 18 and 20. Band 12 has opposing wide surfaces 22 and 24, and opposing end portions 26, 28 each with opposing absolute edges 30 and 32, respectively.

Band 12 is made of a flexible, elastomeric material such as latex and latex-free synthetic rubber. The material is preferably selected to have the same tactile feel as conventional disposable tourniquet bands (or straps). For example, Becton, Dickinson and Co. offers a latex-free disposable tournique under the trade designation “UNIQUET.”

Hemostasis aid 10 preferably has a length and width comparable to that of disposable phlebotomy tourniquets, although other lengths and thicknesses are also suitable. Accordingly, hemostasis aid 10 has a length in the range of about 12 to about 18 inches and a thickness in the range of about ¾ of an inch to about one inch.

As illustrated in FIG. 2, the pressure applicator 14 is preferably a semisphere presenting a flat connecting surface 34 and a rounded contact surface 36. Pressure applicator 14 is generally made of a more rigid material. Pressure applicator 14 may also be constructed of a relatively soft material or include a padding layer for the contact surface provided pressure applicator 14 is relatively more rigid than the surface of the human body targeted for contact, e.g. the arm.

The adhesive 38 for securing pressure applicator 14 to band 12 necessarily varies according to the respective materials of construction. For a latex rubber band and a wooden pressure applicator a polyvinyl adhesive is suitable.

In a preferred embodiment as shown in FIGS. 1-2 and 3-4, pressure applicator 14 is fixed at an end portion 26 of band 12. Surprisingly, it has been found that this special location of the pressure applicator at one end of the elastomeric band is an important feature for gaining acceptance by phlebotomists. The end-point placement of the applicator allows the phlebotomist to apply aid 10 around a patient's limb by first using one hand to press applicator 14 at the puncture site and then with the other hand wrap band 12 over applicator 14 and around the limb.

The requirement for this beneficial effect is that pressure applicator 14,be secured towards the end portion 26. As illustrated in alternate embodiment FIGS. 3 and 4, a short distance of offset from edge 30 is permissible, however.

Hemostasis aid 10 is equipped with a pair of complementary releasably engageable coupling pads 16 and 18 spaced apart and secured to elastomeric band 12. Suitable complementary releasable coupling pads are commercially available from Velcro Industries B.V. (Manchester, NH) and other resellers under the trade designation “VELCRO.” These couplings include one area having a plurality of fibers in a miniloop configuration (i.e. pile) and one area having a plurality of relatively stiffer fibers in a minihook configuration, also referred to herein as a “multiminihook” pad. The minihook area may also be labeled a “burr.”

Hemostasis aid 10 includes a pair of complementary releasably engageable coupling pads in the form of a pile pad 16 and a multiminihook pad 18. As illustrated, hemostasis aid 10 may include a second multiminihook pad 20 for increased adhesion strength. Pile pad 16 and multiminihook pads 18, 20 are preferably secured to band 12 by adhesive, although stitching 21 is also suitable. Furthermore, a combination of stitching and adhesive is also effective.

As shown in FIGS. 1 and 3, pile pad 16 and pressure applicator 14 extend outwardly from the same wide surface side 22 of band 12, while multihook pads 18 and 20 extend outwardly from opposite wide surface side 24.

Pile pad 16 is relatively longer than either multihook pad 18 or 20 (alone or combined) and is made of an elastic material. Suitable elastic pile pads are commercially available from Velcro Industries B.V. under the designation “VELSTRETCH.”

The material for band 12 and pad 16 is selected to provide a hemostasis aid 10 that is elastic in the length direction (i.e. lengthwise) over at least about 80 percent of the length of band 12. More preferably, hemostasis aid 10 is elastic over at least about 90 percent of its length.

Applicant contemplates including advertisement markings on band 12.

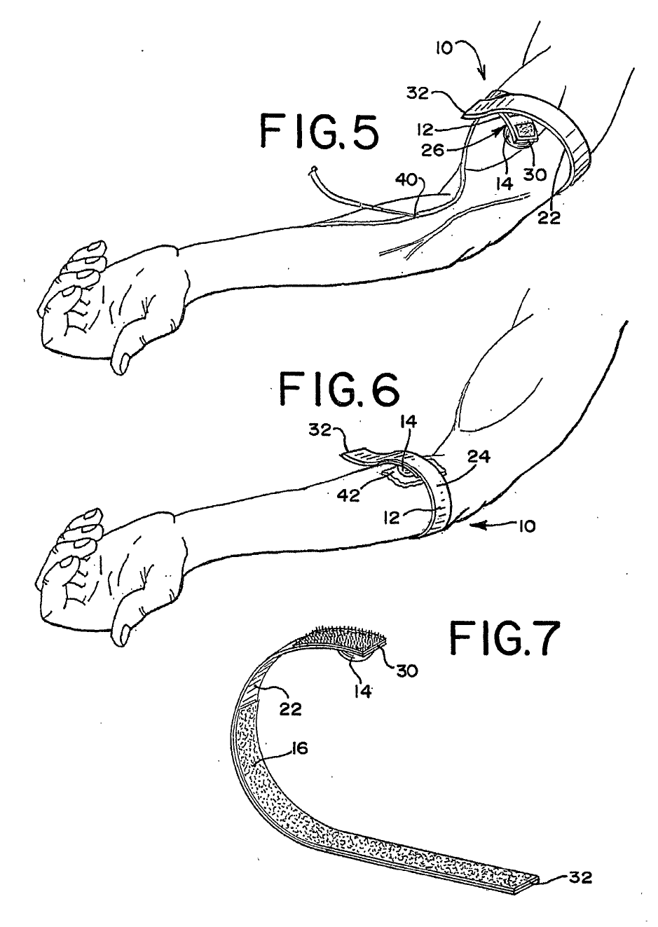

FIGS. 5 and 6 illustrate the use of hemostasis aids according to the present invention. As shown in FIG. 5, hemostasis aid 10 is suitable for use as a phlebotomy tourniquet proximal to the targeted puncture site 40. In the standard tourniquet configuration, ends 26 and 32 of band 12 are overlapped and crossed. Device 10 is elastic and can be stretched to compress the arm tissue and restrict blood flow.

After a blood drawing needle (or other puncture conduit) is withdrawn, the same device (10) can be used to apply pressure to promote hemostasis at the puncture site as shown in FIG. 6. In this hemostasis-aid configuration, side 22 of band 12 and pressure applicator 14 face towards arm. In FIG. 6, gauze 42 or a similar absorbent is positioned between the arm puncture site 40 and applicator 14. Device 10 is elastic and can be stretched around the arm to put compressive force on applicator 14 towards the arm.

FIG. 7 is a perspective view of a hemostasis aid employing a single, relatively longer multiminihook pad 44. Applicator 14 is attached near end 30. Pile pad 16 extends from opposite end 32.

An example embodiment was constructed according to FIGS. 1 and 2 with an open band length (unstretched) of 45.5 centimeters (cm) an open band width of 2.4 centimeters (cm), a pile pad length of 32.5 centimeters (or about 70 percent of the band length) and two multihook pads each 2.0 centimeters (cm) (or about 4 percent each of the band length). An elastic pile pad 16 was attached to band 12 with adhesive and positioned to extend from one of the two opposing ends 28. A semisphere shaped applicator 14 was attached with adhesive to end portion 26 of the band 12 opposite pad 18 such that a multihook pad 18 directly opposes applicator 14 on the opposite surface 24 of band 12. Two multiminihooks pads 18 and 20 were attached by stitching and adhesive.

Users of the example embodiment 10 reported a tactile feedback comparable to a conventional disposable strip tourniquet. Indeed, the medical devices of the present invention have several key features. One key feature of the present invention is substantially uniform elasticity. Another key feature of the present invention is tactile compatibility with conventional disposable tourniquets. The present invention provides a combination tourniquet and hemostasis aid having a lengthwise elasticity substantially similar to universally accepted latex and latex-free disposable tourniquet bands. Through experience, medical personnel are already aware of the necessary or optimal tension with which to wrap limbs at this elasticity.

The foregoing specification and drawings are to be taken as illustrative but not limiting of the present invention. Still other configurations and embodiments utilizing the spirit and scope of the present invention are possible, and will readily present themselves to those skilled in the art.

I claim:

1. A device suitable for inducing blood vessel distension and for reducing post-venipuncture bruising, the device comprising:
an open elastomeric band adapted to be stretched to encircle a limb with overlap;
a pressure applicator secured to the band;
a pair of complementary releasably engageable coupling pads spaced apart and secured to the elastomeric band, and
wherein at least one of the pair of coupling pads is elastic such that the device is lengthwise elastic over at least about 80 percent of the length of the band.

2. The device according to claim 1 wherein the at least 80 percent of the length of the band has a lengthwise elasticity substantially the same as a conventional, disposable rubber tourniquet.

3. The device according to claim 1 wherein the band has a pair of opposing ends and the pressure applicator is mounted substantially at one of the opposing ends.

4. The device according to claim 3 wherein the pressure applicator is offset about 1 centimeter from a remote edge of the opposing end.

5. The device according to claim 1 wherein the band is a latex rubber material.

6. The device according to claim 1 wherein the band is made of a substantially latex-free polymeric material.

7. The device according to claim 1 wherein the applicator is a semisphere.

8. The device according to claim 1 wherein the applicator is secured to the band by an adhesive.

9. The device according to claim 1 wherein each of the pair of complementary releasably engageable coupling pads is secured to the band by an adhesive.

10. The device according to claim 1 wherein each of the pair of complementary releasably engageable coupling pads is secured to the band by stitching.

11. The device according to claim 1 wherein the pair of complementary releasably engageable coupling pads are a multiminihook-to-pile adhesion system.

12. The device according to claim 11 wherein one of the pair of complementary coupling pads is a multiminihook pad opposing the applicator.

13. The device according to claim 1 wherein the band bears advertisement markings.

14. A post-venipuncture bruising control device comprising:
an open elastomeric band has a pair of opposing ends;
a pressure applicator secured to the band at one of the opposing ends; and
a pair of complementary releasably engageable coupling pads spaced apart and secured to the elastomeric band.

15. A post-venipuncture hemostasis aid comprising:
an open elastomeric band including a pair of inter-mating adhesive portions spaced apart over the length of the band;
a pressure applicator extending from the band;
wherein at least one of the pair of adhesive portions is elastic such that the device is elastic over at least about 80 percent of the length of the band.
